(19) United States
(12) Patent Application Publication
(10) Pub. No.: US 2008/0021300 A1
(43) Pub. Date: Jan. 24, 2008

(54) Four-dimensional target modeling and radiation treatment

(76) Inventor: John W. Allison, Los Altos, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/479,358

(22) Filed: Jun. 29, 2006

(51) Int. Cl. A61B 6/00; A61N 5/10

(52) U.S. Cl. 600/407; 378/65; 600/411; 600/427; 600/439

(57) ABSTRACT

A method and apparatus to generate a four-dimensional correlation model for a target region and to develop a radiation treatment plan which includes a relative movement between a target region and a radiation beam path.

### TECHNICAL FIELD

This invention relates to the field of radiotherapy and radiosurgery treatment and, in particular, to treatment planning and delivery.

### BACKGROUND

Pathological anatomies such as tumors and lesions can be treated with an invasive procedure, such as surgery, which can be harmful and full of risks for the patient. A non-invasive method to treat a pathological anatomy (e.g., tumor, lesion, vascular malformation, nerve disorder, etc.) is external beam radiation therapy. In one type of external beam radiation therapy, an external radiation source is used to direct a sequence of x-ray beams at a tumor site from multiple angles, with the patient positioned so the tumor is at the center of rotation (isocenter) of the beam. As the angle of the radiation source changes, every beam passes through the tumor site, but passes through a different area of healthy tissue on its way to the tumor. As a result, the cumulative radiation dose at the tumor is high and the average radiation dose to healthy tissue is low.

The term “radiotherapy” refers to a procedure in which radiation is applied to a target region for therapeutic, rather than necrotic, purposes. The amount of radiation utilized in radiotherapy treatment sessions is typically about an order of magnitude smaller, as compared to the amount used in a radiosurgery session. Radiotherapy is typically characterized by a low dose per treatment (e.g., 100-200 centiGray (cGy)), short treatment times (e.g., 10 to 30 minutes per treatment) and hyperfractionation (e.g., 30 to 45 days of treatment). For convenience, the term “radiation treatment” is used herein to mean radiosurgery and/or radiotherapy unless otherwise noted.

One challenge facing the delivery of radiation to treat pathological anatomies is identifying the target at a particular point in time because the pathological anatomies may move as a function of the patient's breathing or other natural movements. Therefore, in many medical applications, it is useful to accurately track the motion of a moving target region in the human anatomy. For example, in radiation treatment, it is useful to accurately locate and track the motion of a target region due to respiratory or other patient motions during the treatment. Conventional methods and systems have been developed for performing tracking of an internal target region, while measuring and/or compensating for breathing and/or other motions of the patient.

Breath holding and respiratory gating are two primary methods used to compensate for target movement during respiration while a patient is receiving conventional radiation treatments. Breath holding requires the patient to hold his breath at the same point in each breathing cycle, during which time the tumor is treated while it is presumably stationary. A respirometer is often used to measure the tidal volume and ensure the breath is being held at the same location in the breathing cycle during each irradiation moment. This method takes a relatively long time and often requires training the patient to hold his breath in a repeatable manner.

Respiratory gating is the process of turning the radiation beam on and off as a function of the patient's breathing cycle. Respiratory gating does not directly compensate for motions that result from breathing. Rather, radiation treatment is synchronized to the patient's breathing pattern, limiting the radiation beam delivery to times when the tumor is presumably in a reference position. This treatment method may be quicker than the breath holding method, but also may require the patient to have many sessions of training over several days to breathe in the same manner for long periods of time. Conventional respiratory gating also may expose healthy tissue to radiation before or after the tumor passes into the predetermined position. This can add an additional margin of error of about 5-10 mm on top of other margins normally used during treatment.

These conventional methods and systems attempt to correlate internal organ movement with respiration, but are limited by the patient's ability to perform breathing functions in a consistent manner over multiple treatment sessions.

### BRIEF DESCRIPTION OF THE DRAWINGS

The present invention is illustrated by way of example, and not by way of limitation, in the figures of the accompanying drawings.

FIG. 1 illustrates a cross-sectional view of a treatment tracking environment.

FIG. 2 is a graphical representation of an exemplary two-dimensional path of movement of a target region during a respiration period.

FIG. 3 is a graphical representation of an exemplary estimated path for a multi-poly correlation model in two dimensions.

FIGS. 4A-C illustrate another embodiment of an exemplary treatment environment in which a tumor moves relative to a linear accelerator (LINAC).

FIG. 5 is a graphical representation of an exemplary beam timing diagram correlating the beam status to the relative locations of a target region and a critical structure.

FIGS. 6A-C illustrate various embodiments of surface paths that result from application of a radiation beam on a target region over a period of time in which the target region moves relative to the LINAC.

FIG. 7 illustrates one embodiment of a treatment method.

FIG. 8 illustrates one embodiment of a beam control method.

FIG. 9 illustrates one embodiment of a treatment system that may be used to perform radiation treatment in which embodiments of the present invention may be implemented.

FIG. 10 is a schematic block diagram illustrating one embodiment of a treatment delivery system.

FIG. 11 illustrates a three-dimensional perspective view of a radiation treatment process.

### DETAILED DESCRIPTION

The following description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Thus, the specific details set forth are merely exemplary. Particular implementations may vary from these exemplary details and still be contemplated to be within the spirit and scope of the present invention.

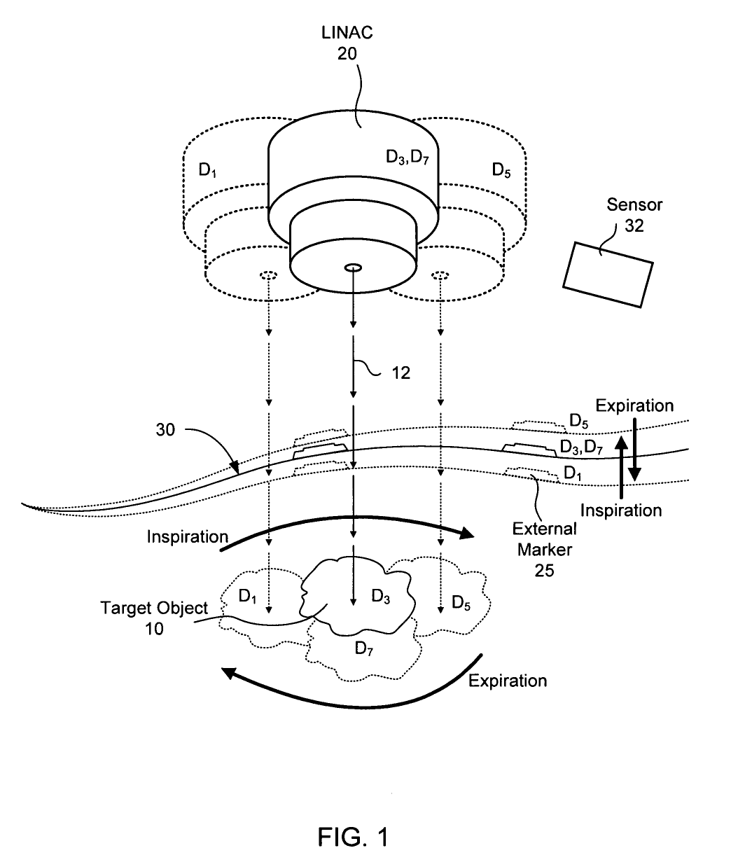

FIG. 1 illustrates a cross-sectional view of a treatment tracking environment. The treatment tracking environment depicts corresponding movements of a target region 10 within a patient, a linear accelerator (LINAC) 20, and an external marker 25. The illustrated treatment tracking environment is representative of a patient chest region, for example, or another region of a patient in which an internal organ or pathological anatomy might move during the respiratory cycle of the patient. In general, the respiratory cycle of a patient will be described in terms of an inspiration interval and an expiration interval, although other designations and/or delineations may be used to describe a respiratory cycle.

In one embodiment, the LINAC 20 moves in one or more dimensions to position and orient itself to deliver a radiation beam 12 to the target 10. Although substantially parallel radiation beams 12 are depicted, the LINAC 20 may move around the patient in multiple dimensions to project radiation beams 12 from several different locations and angles. The LINAC 20 tracks the movement of the target 10 as the patient breathes, for example. One or more external markers 25 are secured to the exterior 30 of the patient in order to monitor the patient's breathing cycle. In one embodiment, the external marker 25 may be a device such as a light source or a metal button attached to a vest worn by the patient. Alternatively, the external marker 25 may be attached to the patient's clothes or skin in another manner. The SYNCHRONY® system, manufactured by Accuray, Inc., is one example of such a tracking system.

As the patient breathes, a tracking sensor 32 tracks the location of the external marker 25. For example, the tracking sensor 32 may track upward movement of the external marker 25 during the inspiration interval and downward movement of the external marker 25 during the expiration interval. The relative position of the external marker 25 is correlated with the location of the target 10, as described below, so that the LINAC 20 may move relative to the location of the external marker 25 and the correlated location of the target 10. In another embodiment, other types of external or internal markers may be used instead of or in addition to the illustrated external marker 25.

As one example, the depicted target 10 is shown in four positions designated as D1, D3, D5, and D7. The first position, D1, may correspond to approximately the beginning of the inspiration interval. The second position, D3, may correspond to a time during the inspiration interval. The third position, D5, may correspond to approximately the end of the inspiration interval and the beginning of the expiration interval. The fourth position, D7, may correspond to a time during the expiration interval. As the patient breathes, the target 10 may move along a path within the patient's body. In one embodiment, the path of the target 10 is asymmetric in that the target 10 travels along different paths during the inspiration and expiration intervals. In another embodiment, the path of the target 10 may be linear or at least partially non-linear. The path of the target 10 may be influenced by the size and shape of the target 10, organs, and tissues surrounding the target 10, the depth or shallowness of the patient's breathing, and so forth. Organs and tissues surrounding the target 10 are also referred to as critical structures, in some cases, to indicate that such structures are not generally targeted for radiation treatment.

Similarly, the external marker 25 is shown in a first position, D1, a second position, D3, a third position, D5, and a fourth position, D7, which correspond to the positions of the target 10. By correlating the positions of the external marker 25 to the target 10, the position of the target 10 may be derived from the position of the external marker 25 even though the external marker 25 may travel in a direction or along a path that is substantially different from the path and direction of the target 10. The LINAC 20 is also shown in a first position, D1, a second position, D3, a third position, D5, and a fourth position, D7, which also correspond to the positions of the target 10. In this way, the movements of the LINAC 20 may be substantially synchronized to the movements of the target 10 as the position of the target 10 is correlated to the sensed position of the external marker 25. Although a specific number of correlated positions are shown in FIG. 1, the LINAC 20 may operate in other positions which also correlate to each of the positions of the external marker 25 and the target 10. For example, the LINAC 20 may be positioned to emit a substantially vertical radiation beam 12 at the target at position D1 during a particular respiratory cycle, and positioned at a distinct location to emit another radiation beam 12 at the target position D1 during a subsequent respiratory cycle.

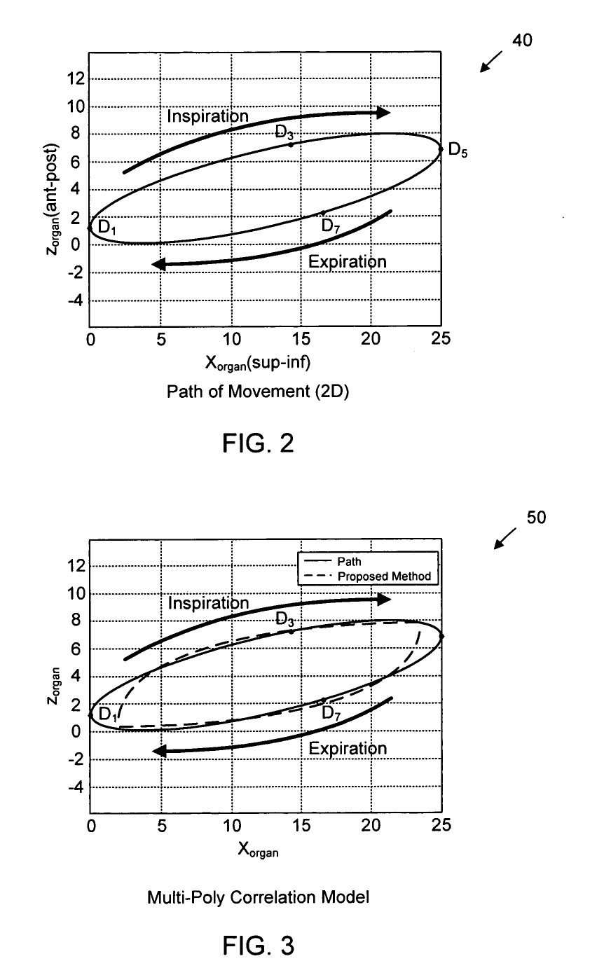

FIG. 2 is a graphical representation 40 of an exemplary two-dimensional path of movement of a target region 10 during a respiration period. The horizontal axis represents displacement (e.g., in millimeters) of the target 10 in a first dimension (x). The vertical axis represents displacement (e.g., in millimeters) of the target 10 in a second dimension (z). The target 10 may similarly move in a third dimension (y). As shown in the graph 40, the path of movement of the target 10 may be non-linear. Additionally, the path of movement may be different during an inspiration period and an expiration period. As an example, the inspiration path may correspond to the upper portion of the graph 40 between zero and twenty-five millimeters in the x direction, with zero being a starting reference position, D1, and twenty-five being the maximum displacement position, D5, at the moment between inspiration and expiration. The corresponding expiration period may be the lower portion of the graph 40 between D5 and D1. In the depicted embodiment, the displacement position D3 is on the inspiration path roughly between D1 and D5. Similarly, the displacement position D7 is on the expiration path roughly between D5 and D1.

FIG. 3 is a graphical representation 50 of an exemplary estimated path for a multi-poly correlation model in two dimensions. The graph 50 superimposes a polynomial correlation model using multiple polynomial approximations for the x and z directions on the path of movement of the target 10, as shown in FIG. 2. In comparison to a linear correlation model or a single polynomial correlation model, the multi-poly correlation model is much more accurate for most, if not all, of the coordinates along the path of movement of the target 10. The illustrated multi-poly correlation model includes two polynomial approximations. Other embodiments may include more than two polynomial approximations. In another embodiment, the multi-poly correlation model also may include one or more linear approximations to approximate a portion of the path of movement or to link the two polynomial approximations together at the moments approximately between inspiration and expiration. In further embodiments, other correlation models, including a single linear correlation model, a multi-linear correlation model, a single polynomial correlation model, or another type of correlation model may be used to model the movement of the target region 10 during a respiratory cycle.

In one embodiment, the locations of the target region 10 at particular moments in time may be determined from two-dimensional or three-dimensional images obtained with an imager such as a CT scanner, and MRI scanner, a PET scanner, an ultrasonic scanner, or another type of scanner. Although a three-dimensional correlation model (i.e., for the x direction, z direction, and time) is shown in the graph 50 of FIG. 3, other correlation models may be defined, including a four-dimensional model. An exemplary four-dimensional model may describe the movement of the target region 10 in the x, y, and z directions (i.e., three-dimensions of space) over time (i.e., fourth dimension). In one embodiment, the four-dimensional model may be constructed of multiple two- or three-dimensional correlation models. In another embodiment, the four-dimensional model may be generated by graphically morphing sequential three-dimensional images together using techniques known in the graphical arts.

The correlation model of FIG. 3 describes the movement of the target region 10 relative to an external marker 25 over a patient's respiratory cycle. However, in another embodiment, the correlation model may be associated with a patient's heartbeat or other rhythmic characteristic other than respiration. Additionally, the correlation model may describe the movement of the target region 10 relative to a skeletal structure, a soft-tissue structure, an internal fiducial marker, or other reference structure or position other than the external marker 25. In another embodiment, the correlation model also may describe a deformation (e.g., expanding, contracting, elongating, etc.) of the target region 10 over time.

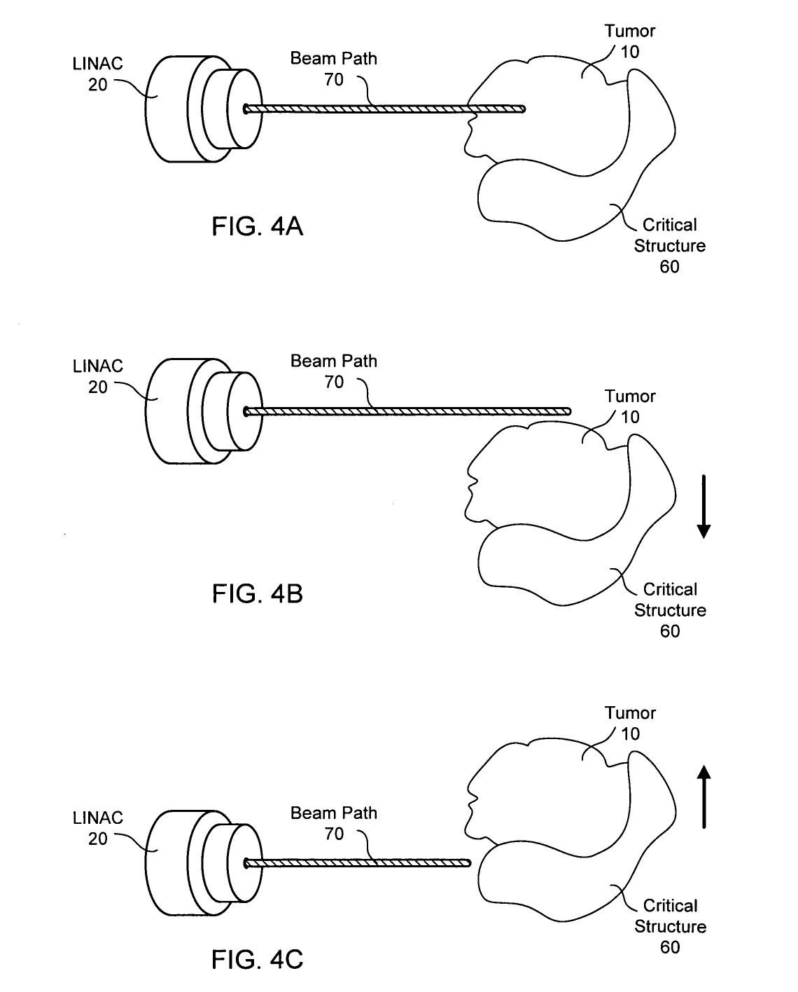

FIGS. 4A-C illustrate another embodiment of an exemplary treatment environment in which a tumor 10 moves relative to a LINAC 20. The depicted tumor 10 is only representative of a target region 10 and other embodiments of the treatment environment may include other types of target regions 10. References to the tumor 10 are understood to refer to a target region 10 generally. The illustrated treatment environment also includes a critical structure 60 that is substantially adjacent to the tumor 10. The critical structure 60 represents some form of physical structure that may be damaged by a radiation beam 12 or is otherwise not intended to receive radiation treatment. Some exemplary critical structures 60 in the human body may include vital organs, soft tissue structures, bone, or other types of structures that may be affected by radiation treatment.

FIG. 4A illustrates a beam path 70 from the LINAC 20. The beam path 70 represents the approximate path that a radiation beam 12 travels if emitted from the LINAC 20 in the depicted position. In FIG. 4A, the beam path 70 intersects with the tumor 10, but does not intersect with the critical structure 60. In FIG. 4B, the beam path 70 does not intersect with the tumor 10 or the critical structure 60. In FIG. 4C, the beam path 70 does not intersect with the tumor 10, but does intersect with the critical structure 60. In certain embodiments, the intersection of the beam path 70 with the tumor 10 or critical structure 60 may result from the relative movement of the LINAC 20 and the tumor 10 or critical structure 60. For example, the LINAC 20 may move from a reference point to align the beam path 70 to intersect with the tumor 10. In another example, the tumor 10 may move as a result of the patient's respiration. In another example, the tumor 10 may move as a result of a physical movement of the patient's body on a treatment couch.

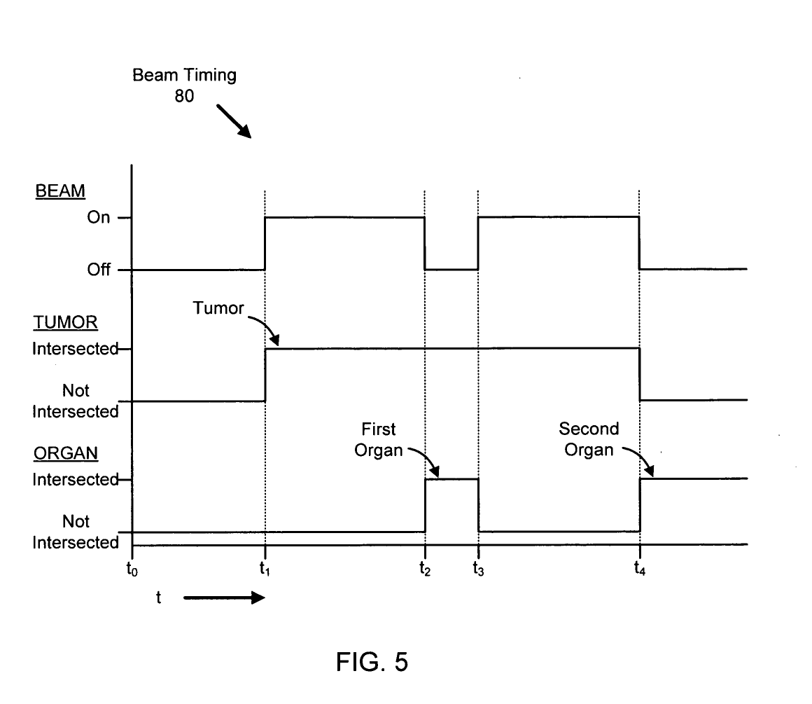

FIG. 5 is a graphical representation of an exemplary beam timing diagram 80 correlating the beam status to the relative locations of a target region 10 and a critical structure 60. The tumor 10 is representative of any target region 10 and an organ 60 is representative of any critical structure 60. In one embodiment, the LINAC 20 operates to apply the radiation beam 12 to the tumor 10, but not to the organ 60.

The first (top) line represents the status of the radiation beam 12. The high position of the first line represents a time when the radiation beam 12 is on. The low position of the first line represents a time when the radiation beam 12 is off. The second (middle) line represents the general position of the target region 10 with respect to the beam path 70. The high position of the second line represents a time when the beam path 70 intersects the target region 10. The low position of the second line represents a time when the beam path 70 does not intersect the target region 10. The third (bottom) line represents the general position of the critical structure 60 with respect to the beam path 70. The high position of the third line represents a time when the beam path 70 intersects the critical structure 60. The low position of the second line represents a time when the beam path 70 intersects the critical structure 60.

In the illustrated beam diagram 80, the radiation beam 12 is on when the beam path 70 intersects the target region 10, but does not intersect the critical structure 60. Otherwise, the radiation beam 12 remains off. For example, the radiation beam 12 is off between times t₀ and t₁ because the beam path 70 does not intersect the target region 10. Embodiments of this relationship are illustrated in FIGS. 4B and 4C, described above. At approximately time t₁, the beam path 70 intersects with at least a portion of the target region 10 and the radiation beam 12 turns on. However, at approximately time t₂, the beam path 70 intersects a first organ 60 and the radiation beam 12 turns off, even though the beam path 70 continues to intersect with the target region 10. Note that the beam path 70 simultaneously may intersect a target region 10 and a critical structure 60 because of the ability of the radiation beam 12 to pass through certain types of target regions 10 or critical structures 60. Then at approximately time t₃, the beam path 70 does not intersect with the first organ 60 and the radiation beam 12 turns on. Subsequently, at time t₄, the beam path 70 does not intersect with the target region 10 and, furthermore, does intersect with a second organ 10 so the radiation beam 12 turns off. Turning the radiation beam 12 on and off in this manner is called gating. One form of gating may be based only on boundaries of a tumor and the intersection of the tumor and surrounding critical structures. In other embodiments, timing of the on and off times of the radiation beam 12 also may be defined to achieve other dose delivery considerations to optimize the dose delivered to a target volume. Some exemplary dose delivery consideration include, but are not limited to, the conformality index, the homogeneity index, and dose volume histograms for target volumes and/or critical structures.

In this way, the LINAC 20 applies the radiation beam 12 to the tumor 10 and not to the critical structures 60, or at least minimizes the radiation exposure of the critical structures 60. Additionally, the radiation beam 12 may remain on during the time in which the beam path 70 intersects with the target region 10, but does not intersect with the critical structure 60. This continuous application of the radiation beam 12 to the target region 10 is referred to as beam sweeping and may have certain advantages compared to other treatments in which the radiation beam 12 is only on for very brief bursts. Additionally, the use of real-time tracking of the target region 10 during treatment delivery may allow more precise application of the radiation beam 12 to the target region 10 and avoidance of the surrounding critical structures 60 compared to a radiation treatment delivery that relies solely on treatment planning performed prior to treatment delivery.

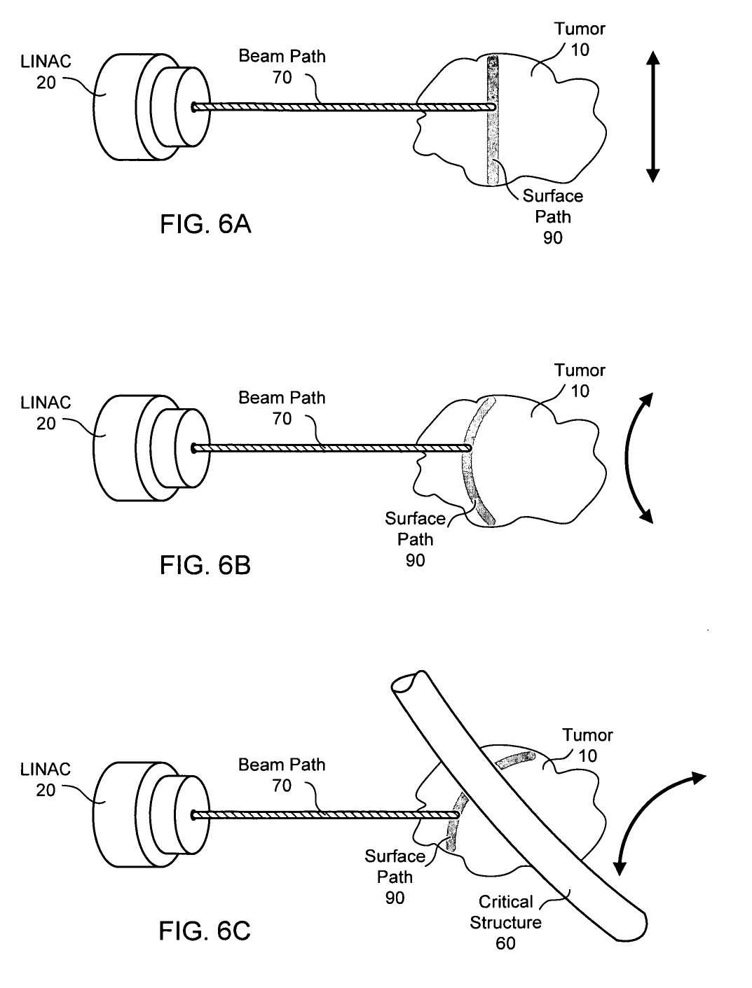

FIGS. 6A-C illustrate various embodiments of surface paths 90 that result from application of a radiation beam 12 on a target region 10 over a period of time in which the target region 10 moves relative to the LINAC 20. As used herein, a surface path 90 is the approximate path defined by the interface of the beam path 70 and the surface of the target region 10. Where a target region 10 does not have a single surface, the interface between the target region 10 and the surrounding tissue may be considered the surface of the target region 10. Additionally, the radiation from the LINAC 20 may penetrate beyond the surface of the target region 10 so the surface path 90, as used herein, is only representative of the radiation applied to the target region 10.

FIG. 6A illustrates a substantially vertical surface path 90 on a tumor 10 that results from a relative movement in the vertical direction (indicated by the arrow) between the tumor 10 and the beam path 70. Although the illustration depicts a simplified vertical line as if the tumor 10 were to have a flat surface, the depicted surface path 90 is only representative of a surface path that may result from a substantially vertical movement of the tumor 10 or LINAC 20. Other surface paths 90 may result from the same or similar relative movement, depending on the locations and orientations of the tumor 10 and LINAC 20, as well as the surface configuration of the tumor 10. FIG. 6B illustrates another surface path 90 on a tumor 10 that may result from another relative movement (indicated by the arrow) between the tumor 10 and the beam path 70.

FIG. 6C illustrates another surface path 90 on a tumor 10 that may result from another relative movement (indicated by the arrow) between the tumor 10 and the beam path 70. The depicted surface path 90 of FIG. 6C illustrates how the radiation beam 12 may be turned off when the radiation beam 70 intersects a critical structure 60. In particular, the LINAC 20 may turn on the radiation beam 12 when the beam path 70 intersects the tumor 10, turn off when the beam path 70 intersects the critical structure 60, and turn on again when the beam path 70 no longer intersects the critical structure 60. In this way, the radiation beam 12 conforms to a modified beam sweeping path that sweeps the radiation beam 12 across the tumor 10, but avoids application of the radiation beam 12 to the intervening critical structure 60.

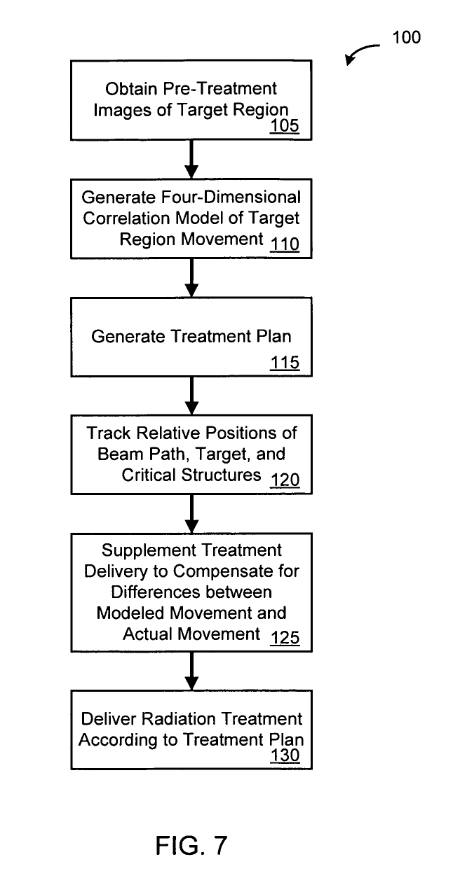

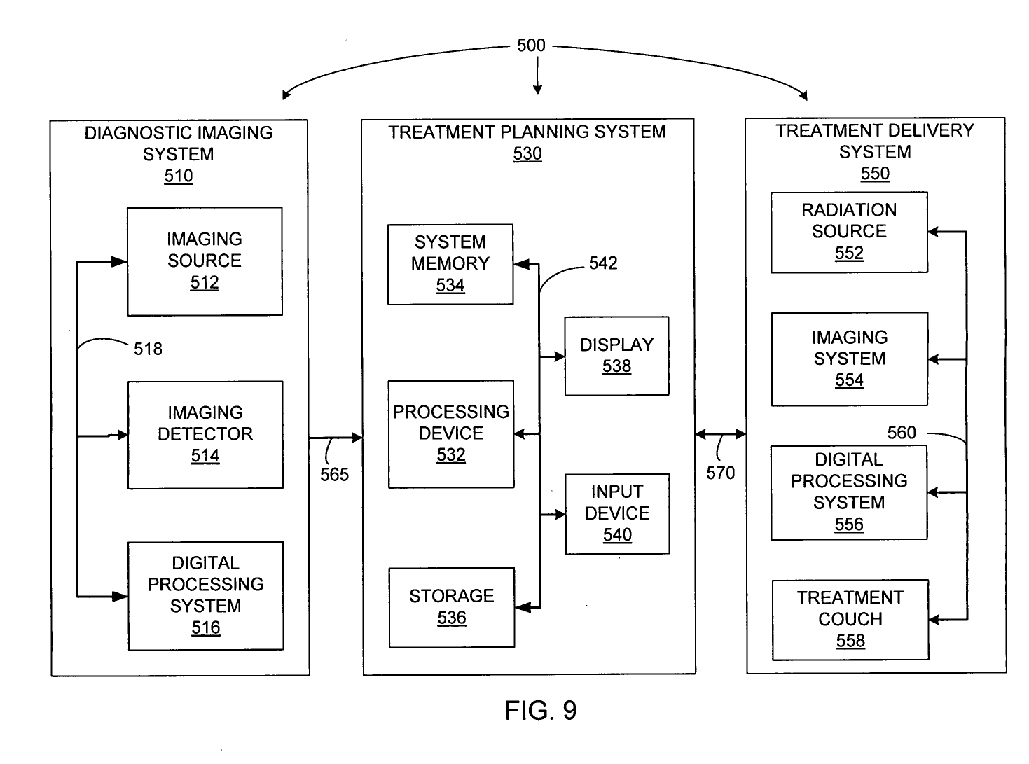

FIG. 7 illustrates one embodiment of a treatment method 100. In one embodiment, the depicted treatment method 100 may be implemented in hardware, software, and/or firmware on a treatment system, such as the treatment system 500 of FIG. 9. Although the treatment method 100 is described in terms of the treatment system 500, or certain parts of the treatment system 500, embodiments of the treatment method 100 may be implemented on another system or independent of the treatment system 500.

The illustrated treatment method 100 begins with the treatment planning phase. In one embodiment, the treatment planning phase may include obtaining 105 pre-treatment images of the target region 10, generating 110 a four-dimensional (4D) correlation model of the movement of the target region 10, and generating 115 a treatment plan. With respect to pre-treatment images, the pre-treatment images may include images of the target region 10 and surrounding tissues, organs, or other structures. In one embodiment, the pre-treatment images may be CT images. However, in alternative embodiments, the pre-treatment images may include other imaging modalities such as MR, PET, and so forth.

The four-dimensional correlation model is generated 110 to correlate the position of the target region 10 relative to the external marker 25. The position of the LINAC 20 relative to the external marker 25 is known or may be calculated at each point in time, thereby correlating the relative positions of the target region 10 and the beam path 70. Using this correlation model, a treatment plan may be generated 110. The treatment plan specifies what radiation dose is to be applied at various time and treatment positions of the LINAC 20. In one embodiment, the treatment plan also may specify beam weighting, beam sweeping, or other treatment attributes.

The treatment planning phase is followed by the treatment delivery phase. In one embodiment, the treatment delivery may include tracking 120 the relative positions of the beam path 70, target region 10, and critical structures 60, supplementing 125 the planned treatment delivery to compensate for differences between the modeled movement and the actual movement of the target region 10 relative to the beam path 70, and delivering 130 radiation treatment to the target region 10.

The supplemental movements implemented to compensate for the differences between the modeled movement and the actual movement may occur prior to or during radiation treatment delivery. During treatment delivery, the relative positions of the external marker 25, target region 10, and beam path 70 are tracked 120 to determine when the beam path 70 intersects with the target region 10. Although the correlation model developed 110 during treatment planning is useful in describing the relative position of the target region 10, the actual conditions during treatment delivery may be slightly different, or the movement of the target region 10 may differ slightly from the movement used to create 110 the correlation model. For example, a patient may breathe more shallowly or more deeply during treatment delivery than during treatment planning. Therefore, tracking 120 the actual location of the target region 10 through periodically imaging the target region 10 during treatment delivery, for example, allows the treatment system 500 to more precisely locate the target region 10 and any surrounding critical structures 60. With the general movement of the target region 10 known from the correlation model and the actual location of the target region 10 known from the delivery tracking, a radiation treatment using beam sweeping or other supplemented movement may be delivered 130 to the target region 10.

In one embodiment, supplementing 125 the planned treatment delivery to compensate for differences between the modeled movement and the actual movement of the target region 10 relative to the beam path 70 may include augmenting the native movement of target region 10 with a combination of movement of the LINAC and/or the patient couch. These augmented movements may compensate for differences between modeled movement and actual movement of the target region 10 and may recreate the movement upon which the four-dimensional treatment plan is based. In another embodiment, radiation treatment delivery begins according to the treatment plan, and the actual relative positions of the target region 10 and critical structures 60 are periodically tracked 120 to update dynamic compensation during the treatment.

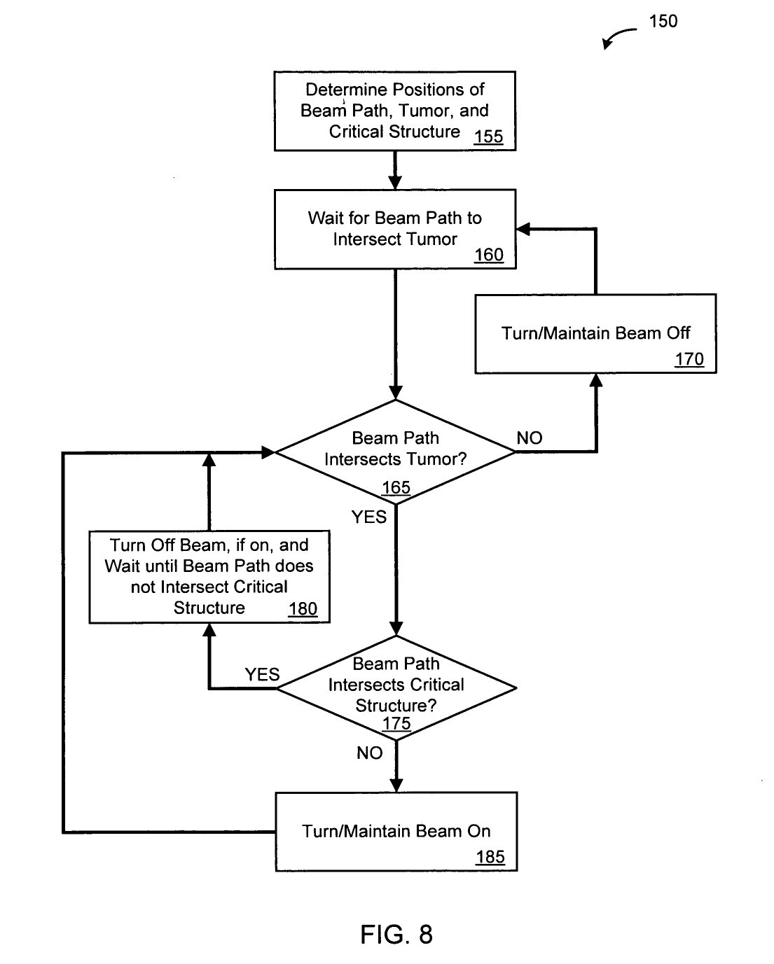

FIG. 8 illustrates one embodiment of a beam control method 150. In one embodiment, the depicted beam control method 150 may be implemented in hardware, software, and/or firmware on a treatment system, such as the treatment system 500 of FIG. 9. Although the beam control method 150 is described in terms of the treatment system 500, or certain parts of the treatment system 500, embodiments of the beam control method 150 may be implemented on another system or independent of the treatment system 500.

The illustrated beam control method 150 begins and the relative positions of the tumor 10 (or other target region 10), critical structure(s) 60, and beam path 70 are determined 155. In one embodiment, the relative positions of the tumor 10, critical structures 60, and beam path 70 may be determined using the four-dimensional correlation model generated 110 during the treatment planning phase. Additionally, the relative positions of the tumor 10, critical structures 60, and beam path 70 may be geometrically calculated with respect to a stationary reference point. In a further embodiment, supplemental movements may be implemented, as described above, to compensate for differences between the modeled and actual positions of the structures.

The LINAC 20 then waits 160 for beam path 70 to intersect the tumor 10. Where a two-dimensional image of the tumor 10 is displayed to an operator, the beam path 70 may or may not intersect with a portion of the tumor 10 displayed in the two-dimensional image. In this way, the time that the beam 12 is on or off is not limited to a single two-dimensional projection of the tumor 70. If the beam path 70 is determined 165 to not intersect the tumor 10, then the LINAC 20 maintains 170 the radiation beam 12 in the off status (or turns the radiation beam 12 off if it was on previously). If the beam path 70 does intersect the tumor 10, but also is determined 175 to intersect a critical structure 60, then the LINAC 20 waits 180 for the critical structure 60 to leave the beam path 70. Otherwise, if the beam path 70 intersects the tumor 10, but does not intersect the critical structure 60, then the LINAC 20 turns on 185 the radiation beam 12 (or maintains the radiation beam 12 on if it was on previously). The beam control method 150 continues is this manner, turning the radiation beam 12 on and off to sweep the tumor 10 according to the alignment of the tumor 10, critical structure(s) 60, and beam path 70, until the treatment session is complete. The depicted beam control method 150 then ends.

Although one embodiment of sweeping a radiation beam 12 across a target region 10 is described, treatment planning does not necessarily assume sweeping of the radiation beam 12 across the target region 10 from one boundary to another. Turning the radiation beam 12 on and off may be based on treatment plan optimization. The native movement of the target region 10, in combination of with synchronized movement of the patient couch and/or LINAC, may result in dose delivery of a particular geometry. For example, delivery of a dose may be in the form of straight lines, pivots about a point inside or outside of the target region 10, curved lines, and stationary point impinging on a portion of the target region 10. A straight line delivers dose in a plane intersecting a portion of the target region 10. A pivot about a point, in which the beam is pivoted about a single point, delivers dose in an hour glass shape with a portion lying within the target region 10. A curved line or arc delivers dose on a complex planar surface intersecting a portion of the target region 10. A stationary point delivers dose along a single line through the target region 10. Additionally, other geometries of dose delivery may be implemented according to the relative movements of the target region 10, LINAC, and patient couch. Furthermore, although gating may be implemented in conjunction with various dose delivery geometries, beam timing also may be implemented to optimize the dosimetry delivered to the target region 10.

FIG. 9 illustrates one embodiment of a treatment system 500 that may be used to perform radiation treatment in which embodiments of the present invention may be implemented. The depicted treatment system 500 includes a diagnostic imaging system 510, a treatment planning system 530, and a treatment delivery system 550. In other embodiments, the treatment system 500 may include fewer or more component systems.

The diagnostic imaging system 510 is representative of any system capable of producing medical diagnostic images of a volume of interest (VOI) in a patient, which images may be used for subsequent medical diagnosis, treatment planning, and/or treatment delivery. For example, the diagnostic imaging system 510 may be a computed tomography (CT) system, a single photon emission computed tomography (SPECT) system, a magnetic resonance imaging (MRI) system, a positron emission tomography (PET) system, a near infrared fluorescence imaging system, an ultrasound system, or another similar imaging system. For ease of discussion, any specific references herein to a particular imaging system such as a CT x-ray imaging system (or another particular system) is representative of the diagnostic imaging system 510, generally, and does not preclude other imaging modalities, unless noted otherwise.

The illustrated diagnostic imaging system 510 includes an imaging source 512, an imaging detector 514, and a digital processing system 516. The imaging source 512, imaging detector 514, and digital processing system 516 are coupled to one another via a communication channel 518 such as a bus. In one embodiment, the imaging source 512 generates an imaging beam (e.g., x-rays, ultrasonic waves, radio frequency waves, etc.) and the imaging detector 514 detects and receives the imaging beam. Alternatively, the imaging detector 514 may detect and receive a secondary imaging beam or an emission stimulated by the imaging beam from the imaging source (e.g., in an MRI or PET scan). In one embodiment, the diagnostic imaging system 510 may include two or more diagnostic imaging sources 512 and two or more corresponding imaging detectors 514. For example, two x-ray sources 512 may be disposed around a patient to be imaged, fixed at an angular separation from each other (e.g., 90 degrees, 45 degrees, etc.) and aimed through the patient toward corresponding imaging detectors 514, which may be diametrically opposed to the imaging sources 514. A single large imaging detector 514, or multiple imaging detectors 514, also may be illuminated by each x-ray imaging source 514. Alternatively, other numbers and configurations of imaging sources 512 and imaging detectors 514 may be used.

The imaging source 512 and the imaging detector 514 are coupled to the digital processing system 516 to control the imaging operations and process image data within the diagnostic imaging system 510. In one embodiment, the digital processing system 516 may communicate with the imaging source 512 and the imaging detector 514. Embodiments of the digital processing system 516 may include one or more general-purpose processors (e.g., a microprocessor), special purpose processors such as a digital signal processor (DSP), or other type of devices such as a controller or field programmable gate array (FPGA). The digital processing system 516 also may include other components (not shown) such as memory, storage devices, network adapters, and the like. In one embodiment, the digital processing system 516 generates digital diagnostic images in a standard format such as the Digital Imaging and Communications in Medicine (DICOM) format. In other embodiments, the digital processing system 516 may generate other standard or non-standard digital image formats.

Additionally, the digital processing system 516 may transmit diagnostic image files such as DICOM files to the treatment planning system 530 over a data link 560. In one embodiment, the data link 560 may be a direct link, a local area network (LAN) link, a wide area network (WAN) link such as the Internet, or another type of data link. Furthermore, the information transferred between the diagnostic imaging system 510 and the treatment planning system 530 may be either pulled or pushed across the data link 560, such as in a remote diagnosis or treatment planning configuration. For example, a user may utilize embodiments of the present invention to remotely diagnose or plan treatments despite the existence of a physical separation between the system user and the patient.

The illustrated treatment planning system 530 includes a processing device 532, a system memory device 534, an electronic data storage device 536, a display device 538, and an input device 540. The processing device 532, system memory 534, storage 536, display 538, and input device 540 may be coupled together by one or more communication channel 542 such as a bus.

The processing device 532 receives and processes image data. The processing device 532 also processes instructions and operations within the treatment planning system 530. In certain embodiments, the processing device 532 may include one or more general-purpose processors (e.g., a microprocessor), special purpose processors such as a digital signal processor (DSP), or other types of devices such as a controller or field programmable gate array (FPGA).

In particular, the processing device 532 may be configured to execute instructions for performing treatment operations discussed herein. For example, the processing device 532 may identify a non-linear path of movement of a target within a patient and develop a non-linear model of the non-linear path of movement. In another embodiment, the processing device 532 may develop the non-linear model based on a plurality of position points and a plurality of direction indicators. In another embodiment, the processing device 532 may generate a plurality of correlation models and select one of the plurality of models to derive a position of the target. Furthermore, the processing device 532 may facilitate other diagnosis, planning, and treatment operations related to the operations described herein.

In one embodiment, the system memory 534 may include random access memory (RAM) or other dynamic storage devices. As described above, the system memory 534 may be coupled to the processing device 532 by the communication channel 542. In one embodiment, the system memory 534 stores information and instructions to be executed by the processing device 532. The system memory 534 also may be used for storing temporary variables or other intermediate information during execution of instructions by the processing device 532. In another embodiment, the system memory 534 also may include a read only memory (ROM) or other static storage device for storing static information and instructions for the processing device 532.

In one embodiment, the storage 536 is representative of one or more mass storage devices (e.g., a magnetic disk drive, tape drive, optical disk drive, etc.) to store information and instructions. The storage 536 and/or the system memory 534 also may be referred to as machine readable media. In a specific embodiment, the storage 536 may store instructions to perform the modeling operations discussed herein. For example, the storage 536 may store instructions to acquire and store data points, acquire and store images, identify non-linear paths, develop linear and/or non-linear correlation models, and so forth. In another embodiment, the storage 536 may include one or more databases.

In one embodiment, the display 538 may be a cathode ray tube (CRT) display, a liquid crystal display (LCD), or another type of display device. The display 538 displays information (e.g., a two-dimensional or three-dimensional representation of the VOI) to a user. The input device 540 may include one or more user interface devices such as a keyboard, mouse, trackball, or similar device. The input device(s) 540 may also be used to communicate directional information, to select commands for the processing device 532, to control cursor movements on the display 538, and so forth.

Although one embodiment of the treatment planning system 530 is described herein, the described treatment planning system 530 is only representative of an exemplary treatment planning system 530. Other embodiments of the treatment planning system 530 may have many different configurations and architectures and may include fewer or more components. For example, other embodiments may include multiple buses, such as a peripheral bus or a dedicated cache bus. Furthermore, the treatment planning system 530 also may include Medical Image Review and Import Tool (MIRIT) to support DICOM import so that images can be fused and targets delineated on different systems and then imported into the treatment planning system 530 for planning and dose calculations. In another embodiment, the treatment planning system 530 also may include expanded image fusion capabilities that allow a user to plan treatments and view dose distributions on any one of various imaging modalities such as MRI, CT, PET, and so forth. Furthermore, the treatment planning system 530 may include one or more features of convention treatment planning systems.

In one embodiment, the treatment planning system 530 may share a database on the storage 536 with the treatment delivery system 550 so that the treatment delivery system 550 may access the database prior to or during treatment delivery. The treatment planning system 530 may be linked to treatment delivery system 550 via a data link 570, which may be a direct link, a LAN link, or a WAN link, as discussed above with respect to data link 560. Where LAN, WAN, or other distributed connections are implemented, any of components of the treatment system 500 may be in decentralized locations so that the individual systems 510, 530, 550 may be physically remote from one other. Alternatively, some or all of the functional features of the diagnostic imaging system 510, the treatment planning system 530, or the treatment delivery system 550 may be integrated with each other within the treatment system 500.

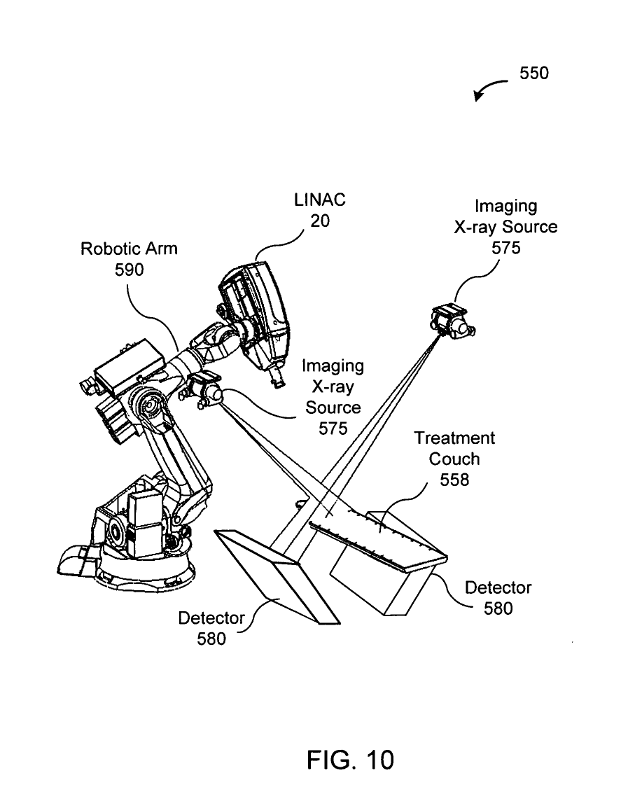

The illustrated treatment delivery system 550 includes a radiation source 552, an imaging system 554, a digital processing system 556, and a treatment couch 558. The radiation source 552, imaging system 554, digital processing system 556, and treatment couch 558 may be coupled to one another via one or more communication channels 560. One example of a treatment delivery system 550 is shown and described in more detail with reference to FIG. 10.

In one embodiment, the radiation source 552 is a therapeutic or surgical radiation source 552 to administer a prescribed radiation dose to a target volume in conformance with a treatment plan. For example, the target volume may be an internal organ, a tumor, a region. As described above, reference herein to the target, target volume, target region, target area, or internal target refers to any whole or partial organ, tumor, region, or other delineated volume that is the subject of a treatment plan.

In one embodiment, the imaging system 554 of the treatment delivery system 550 captures intra-treatment images of a patient volume, including the target volume, for registration or correlation with the diagnostic images described above in order to position the patient with respect to the radiation source. Similar to the diagnostic imaging system 510, the imaging system 554 of the treatment delivery system 550 may include one or more sources and one or more detectors.

The treatment delivery system 550 also may include a digital processing system 556 to control the radiation source 552, the imaging system 554, and a treatment couch 558, which is representative of any patient support device. The digital processing system 556 may include bne or more general-purpose processors (e.g., a microprocessor), special purpose processors such as a digital signal processor (DSP), or other devices such as a controller or field programmable gate array (FPGA). Additionally, the digital processing system 556 may include other components (not shown) such as memory, storage devices, network adapters, and the like.

FIG. 10 is a schematic block diagram illustrating one embodiment of a treatment delivery system 550. The depicted treatment delivery system 550 includes a radiation source 552, in the form of a linear accelerator (LINAC) 20, and a treatment couch 558, as described above. The treatment delivery system 550 also includes multiple imaging x-ray sources 575 and detectors 580. The two x-ray sources 575 may be nominally aligned to project imaging x-ray beams through a patient from at least two different angular positions (e.g., separated by 90 degrees, 45 degrees, etc.) and aimed through the patient on the treatment couch 558 toward the corresponding detectors 580. In another embodiment, a single large imager may be used to be illuminated by each x-ray imaging source 575. Alternatively, other quantities and configurations of imaging sources 575 and detectors 580 may be used. In one embodiment, the treatment delivery system 550 may be an image-guided, robotic-based radiation treatment system (e.g., for performing radiosurgery) such as the CYBERKNIFE® system developed by Accuray Incorporated of California.

In the illustrated embodiment, the LINAC 20 is mounted on a robotic arm 590. The robotic arm 590 may have multiple (e.g., 5 or more) degrees of freedom in order to properly position the LINAC 20 to irradiate a target such as a pathological anatomy with a beam delivered from many angles in an operating volume around the patient. The treatment implemented with the treatment delivery system 550 may involve beam paths with a single isocenter (point of convergence), multiple isocenters, or without any specific isocenters (i.e., the beams need only intersect with the pathological target volume and do not necessarily converge on a single point, or isocenter, within the target). Furthermore, the treatment may be delivered in either a single session (mono-fraction) or in a small number of sessions (hypo-fractionation) as determined during treatment planning. In one embodiment, the treatment delivery system 550 delivers radiation beams according to the treatment plan without fixing the patient to a rigid, external frame to register the intra-operative position of the target volume with the position of the target volume during the pre-operative treatment planning phase.

As described above, the digital processing system 556 may implement algorithms to register images obtained from the imaging system 554 with pre-operative treatment planning images obtained from the diagnostic imaging system 510 in order to align the patient on the treatment couch 558 within the treatment delivery system 550. Additionally, these images may be used to precisely position the radiation source 552 with respect to the target volume or target.

In one embodiment, the treatment couch 558 may be coupled to second robotic arm (not shown) having multiple degrees of freedom. For example, the second arm may have five rotational degrees of freedom and one substantially vertical, linear degree of freedom. Alternatively, the second arm may have six rotational degrees of freedom and one substantially vertical, linear degree of freedom. In another embodiment, the second arm may have at least four rotational degrees of freedom. Additionally, the second arm may be vertically mounted to a column or wall, or horizontally mounted to pedestal, floor, or ceiling. Alternatively, the treatment couch 558 may be a component of another mechanism, such as the AXUM® treatment couch developed by Accuray Incorporated of California. In another embodiment, the treatment couch 558 may be another type of treatment table, including a conventional treatment table.

Although one exemplary treatment delivery system 550 is described above, the treatment delivery system 550 may be another type of treatment delivery system. For example, the treatment delivery system 550 may be a gantry based (isocentric) intensity modulated radiotherapy (IMRT) system, in which a radiation source 552 (e.g., a LINAC 20) is mounted on the gantry in such a way that it rotates in a plane corresponding to an axial slice of the patient. Radiation may be delivered from several positions on the circular plane of rotation. In another embodiment, the treatment delivery system 550 may be a stereotactic frame system such as the GAMMAKNIFE®, available from Elekta of Sweden.

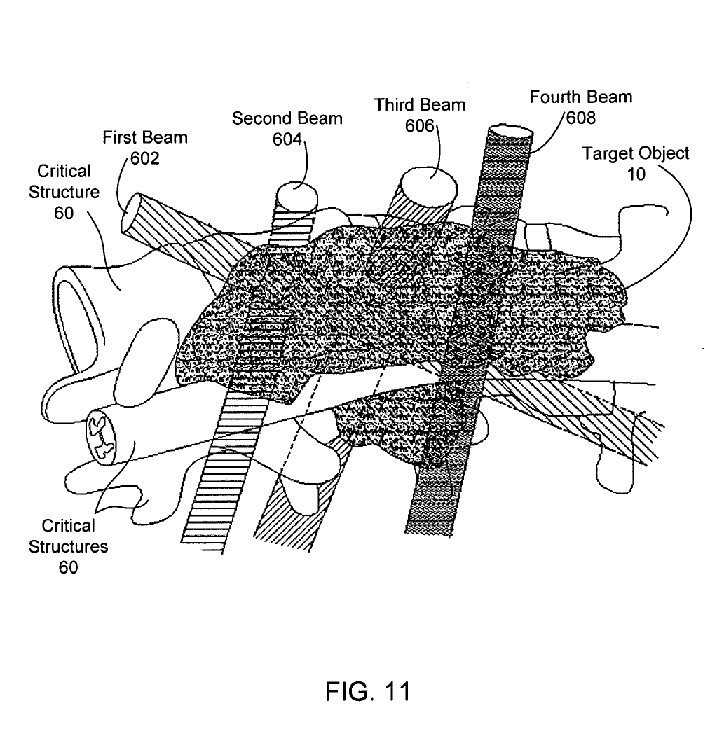

FIG. 11 illustrates a three-dimensional perspective view of a radiation treatment process. In particular, FIG. 11 depicts several radiation beams directed at a target 10. In one embodiment, the target 10 may be representative of an internal organ, a region within a patient, a pathological anatomy such as a tumor or lesion, or another type of object or area of a patient. The target 10 also may be referred to herein as a target region, a target volume, and so forth, but each of these references is understood to refer generally to the target 10, unless indicated otherwise.

The illustrated radiation treatment process includes a first radiation beam 602, a second radiation beam 604, a third radiation beam 606, and a fourth radiation beam 608. Although four radiation beams 12 are shown, other embodiments may include fewer or more radiation beams. For convenience, reference to one radiation beam 12 is representative of all of the radiation beams 12, unless indicated otherwise. Additionally, the treatment sequence for application of the radiation beams 12 may be independent of their respective ordinal designations.

In one embodiment, the four radiation beams 12 are representative of beam delivery based on conformal planning, in which the radiation beams 12 pass through or terminate at various points within target region 10. In conformal planning, some radiation beams 12 may or may not intersect or converge at a common point in three-dimensional space. In other words, the radiation beams 12 may be non-isocentric in that they do not necessarily converge on a single point, or isocenter. However, the radiation beams 12 may wholly or partially intersect at the target 10 with one or more other radiation beams 12.

In another embodiment, the intensity of each radiation beam 12 may be determined by a beam weight that may be set by an operator or by treatment planning software. The individual beam weights may depend, at least in part, on the total prescribed radiation dose to be delivered to target 10, as well as the cumulative radiation dose delivered by some or all of the radiation beams 12. For example, if a total prescribed dose of 3500 cGy is set for the target 10, the treatment planning software may automatically predetermine the beam weights for each radiation beam 12 in order to balance conformality and homogeneity to achieve that prescribed dose. Conformality is the degree to which the radiation dose matches (conforms to) the shape and extent of the target 10 (e.g., tumor) in order to avoid damage to critical adjacent structures. Homogeneity is the uniformity of the radiation dose over the volume of the target 10. The homogeneity may be characterized by a dose volume histogram (DVH), which ideally may be a rectangular function in which 100 percent of the prescribed dose would be over the volume of the target 10 and would be zero everywhere else.

In the depicted embodiment, the various radiation beams 12 are directed at the target region 10 so that the radiation beams 12 do not intersect with the critical structures 60. However, in certain situations it may be acceptable for a number of radiation beams 12 to pass through critical structures 60 in order to realize a determined dose distribution to the target region 10. In such cases, doses may be implemented which are clinically acceptable in accordance with the treatment plan and commonly used dose volume histogram values (DVH). In another embodiment, the radiation beams 12 may deliver radiation treatment to the target region 10 by sweeping across the target region 10, as described above. The beam sweeping radiation treatment may be effectuated or facilitated by the relative movement between the target region 10 and the beam paths 70 of the individual radiation beams 12. Beam sweeping may follow specific paths and geometries as planned for in the treatment plan to achieve a particular dosimetry delivered to the target region 10.

It should be noted that the methods and apparatus described herein are not limited to use only with medical diagnostic imaging and treatment. In alternative embodiments, the methods and apparatus herein may be used in applications outside of the medical technology field, such as industrial imaging and non-destructive testing of materials (e.g., motor blocks in the automotive industry, airframes in the aviation industry, welds in the construction industry and drill cores in the petroleum industry) and seismic surveying. In such applications, for example, “treatment” may refer generally to the application of a beam(s) and “target” may refer to a non-anatomical object or area.

Embodiments of the present invention include various operations, which are described herein. These operations may be performed by hardware components, software, firmware, or a combination thereof. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Certain embodiments may be implemented as a computer program product that may include instructions stored on a machine-readable medium. These instructions may be used to program a general-purpose or special-purpose processor to perform the described operations. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; electrical, optical, acoustical, or other form of propagated signal (e.g., carrier waves, infrared signals, digital signals, etc.); or another type of medium suitable for storing electronic instructions.

Additionally, some embodiments may be practiced in distributed computing environments where the machine-readable medium is stored on and/or executed by more than one computer system. In addition, the information transferred between computer systems may either be pulled or pushed across the communication medium connecting the computer systems such as in a remote diagnosis or monitoring system. In remote diagnosis or monitoring, a user may diagnose or monitor a patient despite the existence of a physical separation between the user and the patient. In addition, the treatment delivery system may be remote from the treatment planning system.

The digital processing device(s) described herein may include one or more general-purpose processing devices such as a microprocessor or central processing unit, a controller, or the like. Alternatively, the digital processing device may include one or more special-purpose processing devices such as a digital signal processor (DSP), an application specific integrated circuit (ASIC), a field programmable gate array (FPGA), or the like. In an alternative embodiment, for example, the digital processing device may be a network processor having multiple processors including a core unit and multiple microengines. Additionally, the digital processing device may include any combination of general-purpose processing device(s) and special-purpose processing device(s).

Although the operations of the method(s) herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner. Additionally, some operations may be repeated within an iteration of a particular method.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative sense rather than a restrictive sense.

What is claimed is:

1. A method, comprising:
developing a four-dimensional model to describe a movement of a target region over time; and
developing a radiation treatment plan based on the four-dimensional model, wherein developing the radiation treatment plan comprises determining a relative movement between a radiation beam path and the target region.

2. The method of claim 1, wherein developing the radiation treatment plan further comprises defining a beam on time, a beam off time, and a relative position of a radiation source to produce a predetermined dose delivery geometry.

3. The method of claim 2, wherein the predetermined dose delivery geometry comprises a line, a point, a pivot, an arc, or a complex geometry.

4. The method of claim 1, wherein developing the radiation treatment plan further comprises:
defining a beam on time when the target region intersects with the radiation beam path during the relative movement between the radiation beam path and the target region; and
defining a beam off time when the target region does not intersect with the radiation beam path.

5. The method of claim 4, further comprising defining the beam off time when the radiation beam path intersects with a critical structure near the target region.

6. The method of claim 5, further comprising delivering radiation treatment to the target region according to the radiation treatment plan.

7. The method of claim 6, further comprising tracking an actual position of the target region relative to the radiation beam path during delivery of the radiation treatment.

8. The method of claim 7, further comprising comparing the actual position of the target region to the four-dimensional model.

9. The method of claim 6, further comprising moving a radiation source, a patient couch, or both to at least partially produce the relative movement between the radiation beam path and the target region.

10. The method of claim 9, wherein the radiation source comprises a gantry radiation source.

11. The method of claim 6, further comprising maintaining a radiation source substantially stationary as the target region moves relative to the radiation source.

12. The method of claim 11, wherein the target region moves relative to the radiation source as a result of a natural movement of a patient in which the target region is located.

13. The method of claim 6, further comprising moving a patient couch on which a patient is located to produce the relative movement between the target region and the radiation beam path, wherein the target region is located in the patient.

14. The method of claim 6, further comprising moving a radiation source relative to the target region, wherein the target region remains substantially stationary.

15. The method of claim 6, further comprising moving a radiation source or a patient couch on which a patient is located or both to produce a canceling movement that is at least partially complementary to the movement of the target region, wherein the target region is located within the patient.

16. The method of claim 15, wherein the canceling movement exhibits up to six degrees of freedom with respect to the target region.

17. A machine readable medium having instructions thereon, which instructions, when executed by a digital processing device, cause the digital processing device to perform the following, comprising:
develop a four-dimensional model to describe a movement of a target region over time; and
develop a radiation treatment plan based on the four-dimensional model, wherein the radiation treatment plan comprises a relative movement between a radiation beam path and the target region.

18. The machine readable medium of claim 17, wherein the radiation treatment plan defines a beam on time when the target region intersects with the radiation beam path during the relative movement between the target region and the radiation beam path, and defines a beam off time when the target region does not intersect with the radiation beam path.

19. The machine readable medium of claim 18, having further instructions thereon, which further instructions, when executed by the digital processing device, cause the digital processing device to perform the following, comprising avoid application of a radiation beam along the radiation beam path to a critical structure near the target region.

20. The machine readable medium of claim 17, having further instructions thereon, which further instructions, when executed by the digital processing device, cause the digital processing device to perform the following, comprising deliver radiation treatment to the target region according to the radiation treatment plan.

21. The machine readable medium of claim 20, having further instructions thereon, which further instructions, when executed by the digital processing device, cause the digital processing device to perform the following, comprising control a radiation source or a patient couch or both to implement a relative movement between the target region and the radiation beam path.

22. The machine readable medium of claim 20, having further instructions thereon, which further instructions, when executed by the digital processing device, cause the digital processing device to perform the following, comprising control a radiation source or a patient couch or both to implement a relatively stationary relationship between the target region and the radiation beam path.

23. An apparatus, comprising:
a processor to generate a four-dimensional model of a target region, and to develop a radiation treatment plan based on the four-dimensional model, wherein the radiation treatment plan comprises a relative movement between a radiation beam path and the target region.

24. The apparatus of claim 23, wherein the processor is further configured to correlate a first position of the target region and a reference position at a first corresponding point in time, and to correlate a second position of the target region and the reference position at a second corresponding point in time, wherein the four-dimensional model correlates a third position of the target region and the reference point at a time between the first and second points in time.

25. The apparatus of claim 23, further comprising an imager to obtain a plurality of three-dimensional images of the target region, each of the plurality of three-dimensional images showing a position of the target region and a reference position at a corresponding point in time.

26. The apparatus of claim 25, wherein the position and the reference position correlate to an identified portion of a periodic anatomical cycle, wherein the periodic anatomical cycle relates to a respiratory cycle or a cardiac cycle.

27. The apparatus of claim 23, wherein the radiation treatment plan defines a beam on time when the target region intersects with the radiation beam path during the relative movement between the target region and the radiation beam path, and defines a beam off time when the target region does not intersect with the radiation beam path.

28. The apparatus of claim 27, wherein the processor is further configured to develop the radiation treatment plan to avoid application of a radiation beam along the radiation beam path to a critical structure near the target region.

29. The apparatus of claim 23, further comprising a radiation source to deliver radiation treatment to the target region according to the radiation treatment plan.

30. The apparatus of claim 29, further comprising a treatment delivery imaging system to track an actual position of the target region relative to the radiation beam path during delivery of the radiation treatment.

31. The system of claim 29, further comprising a patient couch, wherein the processor is further configured to move the radiation source or the patient couch or both to produce the relative movement between the target region and the radiation beam path, wherein the target region is located in a patient.

32. The apparatus of claim 31, wherein the radiation source comprises a linear accelerator (LINAC) mounted to a robotic arm.

33. The apparatus of claim 31, wherein the radiation source comprises a linear accelerator (LINAC) mounted to a gantry.

34. The apparatus of claim 31, further comprising a diagnostic imaging system to generate one or more pre-treatment images of the target region.

35. The apparatus of claim 29, further comprising a patient couch, wherein the processor is further configured to move the radiation source or the patient couch or both to produce a canceling movement that is at least partially complementary to the relative movement between the target region and the radiation source or the patient couch or both.

36. An apparatus, comprising:
means for generating a four-dimensional model of a target region; and
means for developing a radiation treatment plan based on the four-dimensional model, wherein the radiation treatment plan includes a relative movement between a radiation beam path and the target region.

37. The apparatus of claim 36, further comprising:
means for correlating a first position of the target region and a reference position at a first corresponding point in time; and
means for correlating a second position of the target region and the reference position at a second corresponding point in time, wherein the four-dimensional model correlates a third position of the target region and the reference point at a time between the first and second points in time.

38. The apparatus of claim 36, further comprising means for obtaining a plurality of three-dimensional images of the target region, each of the plurality of three-dimensional images showing a position of the target region and a reference position at a corresponding point in time.

39. The apparatus of claim 36, wherein the radiation treatment plan defines a beam on time when the target region intersects with the radiation beam path during the relative movement between the target region and the radiation beam path, and defines a beam off time when the target region does not intersect with the radiation beam path.

40. The apparatus of claim 39, further comprising means for avoiding application of a radiation beam along the radiation beam path to a critical structure near the target region.
